(19) United States
(12) Patent Application Publication
(10) Pub. No.: US 2026/0355727 A1
(43) Pub. Date: Nov. 12, 2026

(54) METHOD FOR PREPARING 4-HYDROXY-2-METHYLENE-BUTANAL, 4-HYDROXY-2-METHYL-BUT-2-ENAL AND ESTERS THEREOF

(71) Applicant: BASF SE, Ludwigshafen am Rhein (DE)

(72) Inventors: Bernd SCHAEFER, Ludwigshafen am Rhein (DE); Jessica Nadine HAMANN, Ludwigshafen am Rhein (DE); Thomas SCHAUB, Ludwigshafen am Rhein (DE); Joaquim Henrique TELES, Ludwigshafen am Rhein (DE)

(21) Appl. No.: 19/105,066

(22) PCT Filed: Aug. 23, 2023
(86) PCT No.: PCT/EP2023/073156

§ 371 Date: Feb. 20, 2025

(30) Foreign Application Priority Data
Aug. 24, 2022 (EP) 22191946.7
Aug. 24, 2022 (EP) 22191954.1

(51) Int. Cl. B01J 31/02; B01J 35/39; C07C 45/33; C07C 67/29

(52) U.S. Cl. CPC B01J 31/0244; B01J 35/39; C07C 45/33; C07C 67/29; B01J 2231/766

(57) ABSTRACT

The present invention relates to a method for preparing 4-hydroxy-2-methylene-butanal, 4-hydroxy-2-methyl-but-2-enal and esters thereof of the formula (I.a) and (I.b) where R¹ is as defined in the claims and the description, by subjecting isoprenol or an ester thereof to a photooxidation in the presence of a photosensitizer and optionally a transition metal catalyst, where in case that photooxidation is carried out without a transition metal catalyst being present, the reaction mixture obtained in the photooxidation is subsequently brought into contact with a transition metal catalyst. The invention relates moreover to certain hydroperoxides of the compounds (I.a) or (I.b); and to the use thereof as intermediates in the synthesis of compounds (I.a) and (I.b) or in the synthesis of retinol, stereoisomers and derivatives thereof.

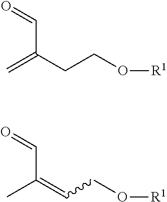

The present invention relates to a method for preparing 4-hydroxy-2-methylene-butanal, 4-hydroxy-2-methyl-but-2-enal and/or esters thereof of the formula I.a and I.b as defined below by subjecting isoprenol or an ester thereof of the formula II.a as defined below to a photooxidation in the presence of a photosensitizer and optionally a transition metal catalyst, where in case that photooxidation is carried out without a transition metal catalyst being present, the reaction mixture obtained in the photooxidation is subsequently brought into contact with a transition metal catalyst. The invention relates moreover to the use of certain compounds of the formula I.a or I.b as defined below as intermediates in the synthesis of retinol, stereoisomers and derivatives, in particular esters, thereof; to certain hydroperoxides of the formula III.a, III.b or III.c as defined below; and to the use thereof as intermediates in the synthesis of compounds I.a and I.b or in the synthesis of retinol, stereoisomers and derivatives, in particular esters, thereof.

### TECHNICAL BACKGROUND

4-Acetoxy-2-methylbut-2-enal (the E isomer of which is also called C₅ acetate), the acetic acid ester of 4-hydroxy-2-methyl-but-2-enal mentioned above, is an important building block in industrial syntheses of retinol, stereoisomers and derivatives thereof. Acetoxy-2-methylbut-2-enal, for example in form of its E-isomer C₅ acetate, is currently obtained on industrial scale from vinylglycol-1,2-diacetate (VGDA), a side product from an industrial process, via hydroformylation and deacetoxylation. The latter steps are described, for example, in DE 10117065 and the references cited therein.

The economic availability of VGDA is however dependent on the unaltered continuation of the industrial process from which it stems. Given the increasing unpredicatbility of the lifespan of such processes, be it because of increasing costs for raw materials and energy or ecological demands or increasingly unreliable supply chains, it is desirable to have alternative routes towards C₅ acetate, isomers and derivatives thereof at hand. Also the limited quantities of VGDA available by said process make alternative routes desirable.

Other known synthetic pathways towards C₅ acetate are the oxidation of prenyl acetate with selenium dioxide, as described, for example, in CN 108997112, the oxidation of benzyl prenyl ether, as described, for example, by S. Inoue et al in Chemistry Lett. 1986, 2035-2038, the oxidation of prenyl chloride with oxygen, as described, for example, in CN 108707076, the oxidation of isoprene, as described, for example, by P. A. Wehrli et al., Synthesis, 1977, 649-650, the acetylation of prenol and the oxidation of the resulting prenol acetate with peroxides under irradiation in the presence of a photosensitizer, as described in CN 110981724 A, and the oxidation of prenol acetate in an electrochemical process, as described in CN 111270261 A. These routes are however not suitable for an application on industrial scale.

4-Acetoxy-2-methylbut-2-enal, the basic alcohol 4-hydroxy-2-methyl-but-2-enal and other esters thereof can be obtained from the respective 2-methylene double bond isomer (i.e. from 3-formylbut-3-enyl acetate, 4-hydroxy-2-methylene-butanal or other esters thereof) by known methods, for example via Pd-catalyzed C—C double bond isomerization as described e.g. in U.S. Pat. No. 4,124,619 or CN 103467287.

It is desirable to find an alternative route to 4-hydroxy-2-methyl-but-2-enal or 4-hydroxy-2-methylene-butanal and esters of these alcohols; ideally, this route should be suitable for an industrial scale. Out of environmental and economic reasons, this route, at least the essential steps thereof, should in particular also work with very low amounts of solvents; ideally neat, i.e. in the absence of solvents.

Isoprenol (3-methylbut-3-en-1-ol) is a bulk chemical readily available from isobutene and formaldehyde. Double bond isomerization thereof leads to prenol (3-methylbut-2-en-1-ol). Esters thereof are obtainable by standard esterification processes.

Photooxidation of alkenes with singlet oxygen to allylic hydroperoxides (Schenck ene reaction) and subsequent dehydration to α-enones have been described in the art.

E. D. Mihelich et al. describe in J. Org. Chem. 1983, 48, 4135-4137 the preparation of α-enones by the reaction of cycloalkenes, methyl oleate and other olefinically unsaturated hydrocarbons with singlet oxygen. To this purpose, oxygen is passed through a reaction mixture containing the olefinically unsaturated hydrocarbon, acetic anhydride, pyridine, N,N-dimethylaminopyridine (DMAP) and tetraphenylporphyrin (TPP) as photosensitizer in methylene chloride, and the reaction is simultaneously irradiated with a sodium vapor lamp.

H.-J. Liu et al. describe in Tetrahedron Lett. 1993, 34(28), 4435-4438 the synthesis of (+)-Qinghaosu. The synthesis encompasses inter alia a step where a tricyclic olefinically unsaturated carbocyclic ring is converted into the corresponding α-enone via irradiation of a reaction mixture containing the unsaturated ring, acetic anhydride, pyridine, DMAP and TPP in methylene chloride through which oxygen is passed.

K. You et al. describe in Journal of Photochemistry and Photobiology A: Chemistry, 2011, 217, 321-325 the photosensitized oxidation of α-pinene, β-pinene and limonene inter alia to α-enones using tetrachlorotetraiodo-fluorescein sodium salt as sensitizer in methanol or DMF as solvents in the presence or absence of lutidine and/or acetic anhydride.

P. Bayer et al. describe in Green Chem., DOI: 10.1039/d0gc00436g the photooxygenation of alkenes with singlet oxygen to hydroperoxides in a solvent-free continuous-flow reaction set-up. The further conversion of the labile hydroperoxides to, for example, α-enones, is described schematically as reaction of the hydroperoxide with acetic anhydride and pyridine in dichloromethane.

E. L. Clennan et al. describe in Photochemistry and Photobiology, 2006, 82, 1226-1232 the photooxidation of various allylic alcohols with singlet oxygen. Inter alia, prenol is converted in the presence of TPP in CDCl₃ to 3-methyl-but-2-enal, 3,3-dimethyloxirane-2-carbaldehyde, 2-hydroperoxy-3-methyl-but-3-en-1-ol and 5,5-dimethyl-1,2-dioxolan-3-ol.

The conversion of acyclic geminally disubstituted α-olefins into aldehydes via photooxidation and dehydration using a transition metal catalyst has not been described in the prior art yet.

### SUMMARY OF THE INVENTION

The present inventors found that 4-hydroxy-2-methyl-but-2-enal, 4-hydroxy-2-methylene-butanal and esters of these alcohols can be obtained by subjecting isoprenol or an ester thereof to a photooxidation in the presence of a photosensitizer and transition metal catalyst, or by subjecting isoprenol or an ester thereof to a photooxidation in the presence of a photosensitizer and subsequently bringing the hydroperoxides formed in the photooxidation into contact with a transition metal catalyst.

The invention thus relates to a method for preparing a compound of the formula I.a or of the formula I.b or a mixture thereof or a stereoisomer of the compound I.a or I.b or a mixture of different stereoisomers of the compound I.a and/or I.b or a mixture of different compounds I.a and/or I.b

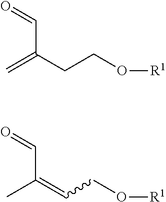

wherein
R¹ is hydrogen or —C(═O)R²; and
R² is C₁-C₂₀-alkyl;
which method comprises
(i) providing a reaction mixture comprising a compound of the formula II.a

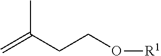

wherein R¹ is as defined above;
a photosensitizer and optionally a transition metal catalyst;
(ii) passing an oxygen-containing gas through the reaction mixture provided in step (i) and simultaneously irradiating the reaction mixture with light;
(iii) if in step (i) no transition metal catalyst has been provided, bringing the reaction mixture of or obtained in step (ii) into contact with a transition metal catalyst;
(iv.1) if desired, after completion of the reaction, isolating the one or more compounds (I.a) or (I.b) obtained in step (ii) or (iii); and
(v.1) if desired hydrolysing the one or more compounds (I.a) or (I.b) isolated in step (iv.1) to compounds (I.a) or (I.b) wherein R¹ is hydrogen;
or
(iv.2) if desired, hydrolysing the reaction mixture obtained in step (ii) or (iii); and
(v.2) if desired, isolating the one or more compounds (I.a) or (I.b) obtained in step (iv.2).

The invention relates moreover to the use of the compound of the formula I.a or I.b different from (E)-4-acetoxy-2-methylbut-2-enal or of a stereoisomer of the compound I.a or I.b different from (E)-4-acetoxy-2-methylbut-2-enal or of a mixture of different stereoisomers of the compound I.a and/or I.b or of a mixture of different compounds I.a and/or I.b as defined above as intermediates in the synthesis of retinol, stereoisomers thereof, derivatives thereof (where the derivatives are in particular esters thereof) or stereoisomers of derivatives thereof (where the derivatives are in particular esters thereof).

The invention relates also to a hydroperoxide compound of the formula III.a, III.b or III.c or a stereoisomer of the compound of the formula III.a, III.b or III.c or a mixture of different stereoisomers of the compound III.a, III.b and/or III.c or a mixture of different compounds III.a, III.b and/or III.c

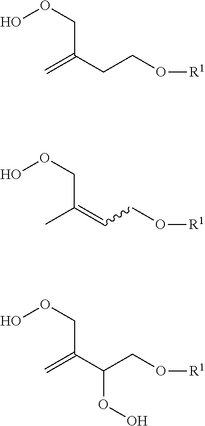

where
in compounds III.a R¹ is hydrogen or —C(═O)R²; and R² is C₁-C₂₀-alkyl;
in compounds III.b R¹ is —C(═O)R²; and R² is C₁-C₂₀-alkyl; and
in compounds III.c R¹ is hydrogen or —C(═O)R²; and R² is C₁-C₂₀-alkyl;
preferably to a hydroperoxide compound of the formula III.a or III.b or a stereoisomer of the compound of the formula III.a or III.b or a mixture of different stereoisomers of the compound III.a and/or III.b or a mixture of different compounds III.a and/or III.b

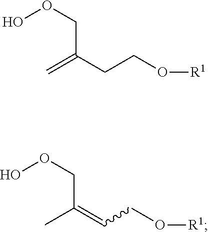

and to the use of said hydroperoxides of the formula III.a, III.b or III.c or of a stereoisomer of the compound of the formula III.a, III.b or III.c or of a mixture of different stereoisomers of the compound III.a, and/or III.b and/or III.c or of a mixture of different compounds III.a, III.b and/or III.c as defined above, where however in compound III.b R¹ can also be hydrogen, preferably of said hydroperoxides of the formula III.a or III.b or of a stereoisomer of the compound of the formula III.a or III.b or of a mixture of different stereoisomers of the compound III.a and/or III.b or of a mixture of different compounds III.a and/or III.b as defined above, where however in compound III.b R¹ can also be hydrogen, as intermediates in the synthesis of compounds of the formula I.a or I.b or of a stereoisomer of the compound I.a or I.b or of a mixture of different stereoisomers of the compound I.a and/or I.b or of a mixture of different compounds I.a and/or I.b as defined above, or as intermediates in the synthesis of retinol, stereoisomers thereof, derivatives thereof (where the derivatives are preferably esters thereof (i.e. retinol esters), retinal or retinoic acid, and are in particular esters thereof) or stereoisomers of derivatives thereof (where the derivatives are preferably esters thereof (i.e. retinol esters), retinal or retinoic acid, and are in particular esters thereof).

### DETAILED DESCRIPTION OF THE INVENTION

### Definitions

“Alkyl” is used in the usual sense. The term “alkyl” refers to saturated straight-chain (linear) or branched hydrocarbon radicals having 1 or 2 (“C₁-C₂-alkyl”), 1 to 4 (“C₁-C₄-alkyl”) or 1 to 20 (“C₁-C₂₀-alkyl”) carbon atoms. C₁-C₂-Alkyl denotes a saturated linear or branched aliphatic acyclic hydrocarbon radical with 1 or 2 carbon atoms. Examples are methyl and ethyl. C₁-C₄-Alkyl denotes a saturated linear or branched aliphatic acyclic hydrocarbon radical with 1 to 4 carbon atoms. Examples are methyl, ethyl, n-propyl, isopropyl, n-butyl, sec-butyl, isobutyl and tert-butyl. C₁-C₂₀-Alkyl denotes a saturated linear or branched aliphatic acyclic hydrocarbon radical with 1 to 20 carbon atoms. Examples are, in addition to those mentioned for C₁-C₄-alkyl, n-pentyl, 1-methylbutyl, 2-methylbutyl, 3-methylbutyl, 2,2-dimethylpropyl, 1-ethylpropyl, 1,1-dimethylpropyl, 1,2-dimethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, n-octyl, 2-ethylhexyl, n-nonyl, n-decyl, 2-propylheptyl, n-undecyl, n-dodecyl, n-tridecyl, n-tetradecyl, n-pentadecyl, n-hexadecyl, n-heptadecyl, n-octadecyl, n-nonadecyl, n-eicosyl and (other) structural isomers thereof. n-C₁₅-Alkyl is CH₃(CH₂)₁₄—.

Chlorinated C₁-C₂-alkanes are methane or ethane in which a part or all of the hydrogen atoms are replaced by chlorine atoms. Examples are dichloromethane (methylene chloride), trichloromethane (chloroform), tetrachloromethane (carbon tetrachloride), 1,1-dichloroethane, 1,2-dichloroethane, 1,1,1-trichloroethane, 1,1,2-trichloroethane, 1,1,1,2-tetrachloroethane, 1,1,2,2-tetrachloroethane and pentachloroethane. C₂-C₈-Carboxylates are the anions or salts of C₂-C₈-carboxylic acids. The anions can be depicted as R—C(═O)O⁻, where R is C₁-C₇-alkyl. Examples are acetate, propionate, butyrate and the like.

The term “stereoisomers” as used in context with the present invention relates to optical isomers, such as enantiomers or diastereomers, the latter existing due to more than one stereogenic center in the molecule, but in particular to Z/E isomers (due to the presence of correspondingly substituted double bonds or ring systems). Thus, stereoisomers of the compounds I.b are primarily the E isomer (E)-I.b and the Z isomer (Z)-I.b:

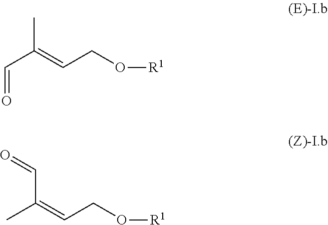

Optical isomers of compounds I.b occur if the radical R¹ is —C(═O)R², and R² is a C₄-C₂₀-alkyl group having one or more stereogenic centers, such as in sec-butyl.

Analogously, stereoisomers of the compounds III.b are primarily the E isomer (E)-III.b and the Z isomer (Z)-III.b:

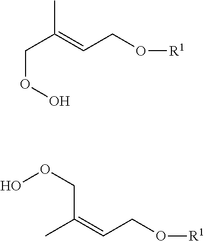

Optical isomers of compounds III.b occur if the radical R¹ is —C(═O)R², and R² is a C₄-C₂₀-alkyl group having one or more stereogenic centers, such as in sec-butyl.

Analogously, optical isomers of compounds I.a occur if the radical R¹ is —C(═O)R², and R² is a C₄-C₂₀-alkyl group having one or more stereogenic centers, such as in sec-butyl; and optical isomers of compounds III.a and III.c occur if the radical R¹ is —C(═O)R², and R² is a C₄-C₂₀-alkyl group having one or more stereogenic centers, such as in sec-butyl.

Mixtures of different stereoisomers of the compounds I.b are primarily mixtures of the E- and the Z-isomer, but can also be mixtures of enantiomers or diastereomers of compounds I.b in which R¹ is —C(═O)R², and R² is a C₄-C₂₀-alkyl group having one or more stereogenic centers. Analogously, mixtures of different stereoisomers of the compounds III.b are primarily mixtures of the E- and the Z-isomer, but can also be mixtures of enantiomers or diastereomers of compounds III.b in which R¹ is —C(═O)R², and R² is a C₄-C₂₀-alkyl group having one or more stereogenic centers. Mixtures of different stereoisomers of the compounds I.a are mixtures of enantiomers or diastereomers of compounds I.a in which R¹ is —C(═O)R², and R² is a C₄-C₂₀-alkyl group having one or more stereogenic centers. Analogously, mixtures of different stereoisomers of the compounds III.a are mixtures of enantiomers or diastereomers of compounds III.a in which R¹ is —C(═O)R², and R² is a C₄-C₂₀-alkyl group having one or more stereogenic centers. Analogously, mixtures of different stereoisomers of the compounds III.c are mixtures of enantiomers or diastereomers of compounds III.c in which R¹ is —C(═O)R², and R² is a C₄-C₂₀-alkyl group having one or more stereogenic centers.

Mixtures of the compounds I.a or I.b can be mixtures of two or more different compounds I.a, the compounds I.a present in the mixture differing in the radical R¹; mixtures of two or more different compounds I.b, the compounds I.b present in the mixture differing in the radical R¹; mixtures of a compound I.a and a compound I.b, where in compounds I.a and I.b the radical R¹ has the same meaning; mixtures of a compound I.a and a compound I.b, where in compounds I.a and I.b the radical R¹ has different meanings; mixtures of a compound I.a with two or more different compounds I.b; mixtures of a compound I.b with two or more different compounds I.a; or mixtures of two or more different compounds I.a with two or more different compounds I.a. Primarily, however, mixtures of the compounds I.a or I.b refers to mixtures of a compound I.a and a compound I.b, where in compounds I.a and I.b the radical R¹ has the same meaning. Compounds I.b in the above-defined mixtures can be present as the pure E isomer, the pure Z isomer or a mixture of the E and Z isomers.

The analogous definition applies to mixtures of the compounds III.a, III.b or III.c.

A photosensitizer in terms of the present invention is an organic molecule (generally a dye) which, when subjected to irradiation (generally to electromagnetic radiation in the UV, in the visible or in the near IR region) can convert triplet oxygen to singlet oxygen: Upon irradiation, the sensitizer forms the corresponding excited singlet state. Intersystem crossing affords the excited triplet state of the sensitizer, thus transferring energy to triplet oxygen to form singlet oxygen.

“Light” in the proper sense is electromagnetic radiation with a wavelength (range) in the visible spectrum (380 to 780 nm). However, in terms of the present invention, unless specified otherwise, the term “light” also encompasses the directly adjacent wavelength spectrum, i.e. near IR (>780 nm to 1 μm) and near UV (315 to <380 nm).

Retinol is (2E,4E,6E,8E)-3,7-dimethyl-9-(2,6,6-trimethylcyclohex-1-enyl)nona-2,4,6,8-tetraen-1-ol (all-trans). Stereoisomers of retinol in terms of the present invention relate to retinol, in which however one, two, three or all four of the double bonds in the 2-, 4-, 6- and 8-position(s) has/have Z geometry. Specific examples for such stereoisomers are:
(2Z,4E,6E,8E)-3,7-dimethyl-9-(2,6,6-trimethylcyclohex-1-enyl)nona-2,4,6,8-tetraen-1-ol;
(2E,4Z,6E,8E)-3,7-dimethyl-9-(2,6,6-trimethylcyclohex-1-enyl)nona-2,4,6,8-tetraen-1-ol;
(2Z,4Z,6E,8E)-3,7-dimethyl-9-(2,6,6-trimethylcyclohex-1-enyl)nona-2,4,6,8-tetraen-1-ol;
or (2Z,4E,6Z,8E)-3,7-dimethyl-9-(2,6,6-trimethylcyclohexen-1-yl)nona-2,4,6,8-tetraen-1-ol (also known as (13Z) retinol under carotenoid nomenclature).

Retinol derivatives in terms of the present invention are preferably retinol esters, i.e. retinol in which the —OH group is esterified to a group —O—C(═O)R, where R is an organic moiety, and is preferably R². Retinol derivatives are however also oxidized forms of retinol, such as retinal (—CH₂OH group is oxidized to —CHO) or retinoic acid (—CH₂OH group is oxidized to —C(═O)OH).

Stereoisomers of retinol derivatives are retinol derivatives as defined above, in which however one, two, three or all four of the double bonds in the 2-, 4-, 6- and 8-position(s) has/have Z geometry.

### Embodiments (E.x) of the Invention

General and preferred embodiments E.x are summarized in the following, non-exhaustive list. Further preferred embodiments become apparent from the paragraphs following this list.
E.1. A method for preparing a compound of the formula I.a or of the formula I.b or a mixture thereof or a stereoisomer of the compound I.a or I.b or a mixture of different stereoisomers of the compound I.a and/or I.b or a mixture of different compounds I.a and/or I.b

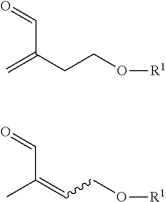

wherein
R¹ is hydrogen or —C(═O)R²; and
R² is C₁-C₂₀-alkyl;
which method comprises
(i) providing a reaction mixture comprising a compound of the formula II.a

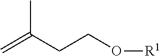

wherein R¹ is as defined above;
a photosensitizer and optionally a transition metal catalyst;
(ii) passing an oxygen-containing gas through the reaction mixture provided in step (i) and simultaneously irradiating the reaction mixture with light;
(iii) if in step (i) no transition metal catalyst has been provided, bringing the reaction mixture of or obtained in step (ii) into contact with a transition metal catalyst;
(iv.1) if desired, after completion of the reaction, isolating the one or more compounds (I.a) or (I.b) obtained in step (ii) or (iii); and
(v.1) if desired hydrolysing the one or more compounds (I.a) or (I.b) isolated in step (iv.1) to compounds (I.a) or (I.b) wherein R¹ is hydrogen;
or
(iv.2) if desired, hydrolysing the reaction mixture obtained in step (ii) or (iii); and
(v.2) if desired, isolating the one or more compounds (I.a) or (I.b) obtained in step (iv.2).
E.2. The method according to embodiment E.1, where R² is C₁-C₄-alkyl or n-C₁₅-alkyl.
E.3. The method according to embodiment E.2, where R² is C₁-C₄-alkyl.
E.4. The method according to embodiment E.3, where R² is methyl.
E.5. The method according to any of the preceding embodiments, where in the compound II.a R¹ is —C(═O)R².
E.6. The method according to any of the preceding embodiments, where the transition metal catalyst comprises transition metal of group 4 to 12 of the Periodic Table of Elements in elemental or oxidized form.
E.7. The method according to embodiment E.6 where the transition metal catalyst comprises a transition metal of the 4^{th} period of group 4 to 12 of the Periodic Table of Elements in elemental or oxidized form.
E.8. The method according to any of the preceding embodiments, where the transition metal catalyst used in step (i) or (iii) is a heterogeneous catalyst.
E.9. The method according to embodiment E.7, where the heterogeneous transition metal catalyst is a supported catalyst.
E.10. The method according to embodiment E.8, where the heterogeneous transition metal catalyst comprises at least one salt or oxide of a transition metal of group 4 to 12 of the Periodic Table of Elements supported on a support material.
E.11. The method according to embodiment E.10 where the salt or oxide of the transition metal is a C₂-C₈-carboxylate, halide, nitrate, phosphate, sulfate, chlorate, perchlorate or oxide of a transition metal of the 4^{th} period of group 4 to 12 of the Periodic Table of Elements, or is a chromate, chlorochromate, dichromate or permanganate.
E.12. The method according to embodiment E.11, where the salt or oxide of the transition metal is a C₂-C₈-carboxylate, halide, nitrate, phosphate, sulfate, chlorate, perchlorate, or oxide of Cu, Cr, Fe or Ni, or is a chromate or dichromate.
E.13. The method according to embodiment E.12, where the chromate or dichromate is an alkali metal, Zn or Fe chromate or dichromate.
E.14. The method according to any of embodiments E.12 or E.13, where the salt or oxide of the transition metal is selected from the group consisting of Cu(II) acetate, Cu(ClO₄)₂, CuCl₂, CuO, Co(II) acetate, CrO₃ and an alkali metal dichromate.
E.15. The method according to embodiment E.14, where the alkali metal dichromate is K₂Cr₂O₇.
E.16. The method according to embodiment E.14 where the salt or oxide of the transition metal is Cr(VI) oxide (CrO₃).
E.17. The method according to any of embodiments E.9 to E.16, where the support material is selected from the group consisting of carbon, such as activated carbon, alumina, silica, silicon carbide, alumosilicates, such as zeolites, titanium dioxide, zirconium dioxide and organic polymers.
E.18. The method according to embodiment E.17, where the support material is an organic polymer.
E.19. The method according to embodiment E.18, where the support material is an organic polymer selected from the group consisting of vinylpyridine homo- and copolymers, N-vinylpyrrolidine homo- and copolymers, styrene homo- and copolymers, polyurethanes, acrylate homo- and copolymers and methacrylate homo- and copolymers.
E.20. The method according to embodiment E.19, where the organic polymer is selected from the group consisting of 2-vinylpyridine homo- and copolymers, 4-vinylpyridine homo- and copolymers and N-vinylpyrrolidine homo- and copolymers.
E.21. The method according to embodiment E.20, where the organic polymer is selected from the group consisting of poly(2-vinylpyridine), poly(4-vinylpyridine), copolymers, preferably block copolymers, of 2-vinylpyridine and methyl methacrylate; and poly(N-vinylpyrrolidine)
E.22. The method according to embodiment E.21, where the organic polymer is selected from the group consisting of polyvinylpyridines, preferably from poly(2-vinylpyridine) and poly(4-vinylpyridine).
E.23. The method according to embodiment E.22, where the organic polymer is selected from the group consisting of poly(4-vinylpyridine).
E.24. The method according to any of the preceding embodiments, where the photosensitizer is selected from the group consisting of fluorescein, eosin, rose bengal, erythrosine, tetraphenylporphyrin, cobalt-tetraphenylporphyrin, zinc-tetraphenylporphyrin, hematoporphyrin, rhodamine B, basacryl brilliant red, methyl violet, methylene blue, fullerene C₆₀, fullerene C₇₀, graphene, carbon nanotubes, Ru(bpy)₃²⁺ salts, Ru(phen)₃²⁺ salts, cercosporin, hypocrellin-A and mixtures thereof.
E.25. The method according to embodiment E.24, where the photosensitizer is selected from the group consisting of tetraphenylporphyrin, cobalt-tetraphenylporphyrin, zinc-tetraphenylporphyrin, methylene blue, Ru(bpy)₃²⁺ salts and Ru(phen)₃²⁺ salts.
E.26. The method according to embodiment E.25, where the photosensitizer is selected from the group consisting of tetraphenylporphyrin, zinc-tetraphenylporphyrin and Ru(bpy)₃²⁺ salts.
E.27. The method according to embodiment E.26, where the photosensitizer is tetraphenylporphyrin.
E.28. The method according to any of the preceding embodiments, where in step (ii) further photosensitizer is added if this is depleted during irradiation.
E.29. The method according to any of the preceding embodiments, where the photosensitizer is used in an overall amount of from 0.00001 to 1 mol-%, relative to 1 mol of the compound of the formula II.a.
E.30. The method according to embodiment E.29, where the photosensitizer is used in an overall amount of from 0.0001 to 0.5 mol-%, relative to 1 mol of the compound of the formula II.a.
E.31. The method according to any of embodiments E.1 to E.28, where the photosensitizer is used in an overall amount of from 0.000005 to 0.01 mol per mol of the compound of the formula II.a.
E.32. The method according to embodiment E.31, where the photosensitizer is used in an overall amount of from 0.00001 to 0.005 mol per mol of the compound of the formula II.a.
E.33. The method according to embodiment E.32, where the photosensitizer is used in an overall amount of from 0.00001 to 0.001 mol per mol of the compound of the formula II.a.
E.34. The method according to any of the preceding embodiments, where in step (ii) the reaction mixture is irradiated with light in the wavelength range of from 350 to 800 nm.
E.35. The method according to embodiment E.34, where in step (ii) the reaction mixture is irradiated with light in the wavelength range of from 350 to 680 nm.
E.36. The method according to embodiment E.35, where in step (ii) the reaction mixture is irradiated with light in the wavelength range of from 400 to 650 nm.
E.37. The method according to embodiment E.36, where in step (ii) the reaction mixture is irradiated with light in the wavelength range of from 400 to 580 nm.
E.38. The method according to embodiment E.37, where in step (ii) the reaction mixture is irradiated with light in the wavelength range of from 400 to 500 nm.
E.39. The method according to any of the preceding embodiments, where in step (ii) the reaction mixture is irradiated with monochromatic light.
E.40. The method according to embodiment E.39, where irradiation in step (ii) is carried out using a monochromatic light source, preferably an electroluminescent lighting device emitting monochromatic light, where at least 90% of the light emitted by said monochromatic light source is in the wavelength range of from 350 to 800 nm.
E.41. The method according to embodiment E.40, where at least 90% of the light emitted by said monochromatic light source is in the wavelength range of from 350 to 680 nm
E.42. The method according to embodiment E.41, where at least 90% of the light emitted by said monochromatic light source is in the wavelength range of from 400 to 650 nm.
E.43. The method according to embodiment E.42, where at least 90% of the light emitted by said monochromatic light source is in the wavelength range of from 400 to 580 nm.
E.44. The method according to embodiment E.43, where at least 90% of the light emitted by said monochromatic light source is in the wavelength range of from 400 to 500 nm.
E.45. The method according to any of embodiments E. 25 to 43, where the photosensitizer is tetraphenylporphyrin, cobalt-tetraphenylporphyrin or zinc-tetraphenylporphyrin and in step (ii) the reaction mixture is irradiated with light in the wavelength range of from 400 to 430 nm, preferably 400 to 420 nm, e.g. 400 to 410 nm; or the photosensitizer is methylene blue and in step (ii) the reaction mixture is irradiated with light in the wavelength range of from 600 to 620 nm, or the photosensitizer is a Ru(bpy)₃²⁺ salt or a Ru(phen)₃²⁺ salt and in step (ii) the reaction mixture is irradiated with light in the wavelength range of from 450 to 480 nm, preferably from 460 to 475 nm.
E.46. The method according to any of embodiments E.39 to E.45, where irradiation in step (ii) is carried out using an electroluminescent lighting device emitting monochromatic light, where the electroluminescent lighting device consists of at least one LED.
E.47. The method according to any of the preceding embodiments, where the oxygen-containing gas used in step (ii) is selected from the group consisting of oxygen, air and mixtures of oxygen and nitrogen containing oxygen in a range of from 1 to 99% by weight, relative to the total weight of the mixture.
E.48. The method according to embodiment E.47, where the oxygen-containing gas used in step (ii) is oxygen.
E.49. The method according to any of the preceding embodiments, where steps (ii) and (iii) are carried out neat.
E.50. The method according to any of embodiments E.1 to E.48, where steps (ii) and (iii) are carried out in the presence of a chlorinated C₁-C₂-alkane, where the molar ratio of the compound (II.a) provided in step (i) to the chlorinated C₁-C₂-alkane is of from 50:1 to 1:1.5.
E.51. The method according to embodiment E.50, where the molar ratio of the compound (II.a) provided in step (i) to the chlorinated C₁-C₂-alkane is of from 30:1 to 1:1, preferably from 20:1 to 1:1.
E.52. The method according to embodiment E.51, where the molar ratio of the compound (II.a) provided in step (i) to the chlorinated C₁-C₂-alkane is of from 15:1 to 2:1.
E.53. The method according to any of embodiments E.50 to E.52, where the chlorinated C₁-C₂-alkane is trichloromethane or tetrachloromethane.
E.54. The method according to any of the preceding embodiments, where step (ii) is carried out at a temperature of from −20 to 150 °C
E.55. The method according to embodiment E.54, where step (ii) is carried out at a temperature of from 0 to 70 °C, preferably from 10 to 60 °C.
E.56. The method according to embodiment E.55 where step (ii) is carried out at a temperature of from 20 to 50 °C.
E.57. The method according to any of the preceding embodiments, where step (ii) is carried out at a pressure of from atmospheric pressure to 100 bar (10 MPa).
E.58. The method according to embodiment E.57, where step (ii) is carried out at a pressure of from atmospheric pressure to 10 bar (1 MPa).
E.59. The method according to any of the preceding embodiments, where in step (ii) either the complete reaction mixture or only a distinct portion of the reaction mixture is irradiated.
E.60. The method according to any of the preceding embodiments, where step (ii) is carried out in a side-loop photoreactor, a continuous flow-photoreactor or a submersible photoreactor.
E.61. The method according to any of the preceding embodiments, where step (ii) is carried out in a reactor comprising a reaction zone for photooxidation and a reaction zone comprising the transition metal catalyst.
E.62. The method according to any of the preceding embodiments, comprising
   (i) providing a reaction mixture comprising a compound of the formula II.a, a photosensitizer and a transition metal catalyst;
   (ii) passing an oxygen-containing gas through the reaction mixture provided in step (i) and simultaneously irradiating the reaction mixture with light;
   (iv.1) if desired, after completion of the reaction, isolating the one or more compounds (I.a) or (I.b) obtained in step (ii); and
   (v.1) if desired hydrolysing the one or more compounds (I.a) or (I.b) isolated in step (iv.1) to compounds (I.a) or (I.b) wherein R¹ is hydrogen;
   or
   (iv.2) if desired, hydrolysing the reaction mixture obtained in step (ii); and
   (v.2) if desired, isolating the one or more compounds (I.a) or (I.b) obtained in step (iv.2).
E.63. The method according to any of embodiments E.1 to E.61, comprising
   (i) providing a reaction mixture comprising a compound of the formula I.a and a photosensitizer;
   (ii) passing an oxygen-containing gas through the reaction mixture provided in step (i) and simultaneously irradiating the reaction mixture with light;
   (iii) adding a transition metal catalyst to the reaction mixture obtained in step (ii);
   (iv.1) if desired, after completion of the reaction, isolating the one or more compounds (I.a) or (I.b) obtained in step (iii); and
   (v.1) if desired hydrolysing the one or more compounds (I.a) or (I.b) isolated in step (iv.1) to compounds (I.a) or (I.b) wherein R¹ is hydrogen;
   or
   (iv.2) if desired, hydrolysing the reaction mixture obtained in step (iii); and
   (v.2) if desired, isolating the one or more compounds (I.a) or (I.b) obtained in step (iv.2).
E.64. The method according to any of embodiments E.1 to E.61, comprising
   (i) providing a reaction mixture comprising a compound of the formula II.a and a photosensitizer;
   (ii) passing an oxygen-containing gas through the reaction mixture provided in step (i) and simultaneously irradiating the reaction mixture with light in a first reaction zone;
   (iii) passing the reaction mixture obtained in step (ii) through a second reaction zone comprising a transition metal catalyst;
      and if desired recirculating the reaction mixture obtained in step (iii) to the first and the second reaction zones once or several times;
   (iv.1) if desired, after completion of the reaction, isolating the one or more compounds (I.a) or (I.b) obtained in step (ii); and
   (v.1) if desired hydrolysing the one or more compounds (I.a) or (I.b) isolated in step (iv.1) to compounds (I.a) or (I.b) wherein R¹ is hydrogen;
   or
   (iv.2) if desired, hydrolysing the reaction mixture obtained in step (iii); and
   (v.2) if desired, isolating the one or more compounds (I.a) or (I.b) obtained in step (iv.2).
E.65. The use of the compound of the formula I.a or I.b different from (E)-4-acetoxy-2-methylbut-2-enal or of a stereoisomer of the compound I.a or I.b different from (E)-4-acetoxy-2-methylbut-2-enal or of a mixture of different stereoisomers of the compound I.a and/or I.b or of a mixture of different compounds I.a and/or I.b as defined in any of embodiments E.1 to E.5, as intermediates in the synthesis of retinol, stereoisomers thereof, derivatives thereof (preferably esters thereof) or stereoisomers of derivatives thereof (preferably stereoisomers of esters thereof).
E.66. A hydroperoxide compound of the formula III.a, III.b or III.c or a stereoisomer of the compound of the formula III.a, III.b or III.c or a mixture of different stereoisomers of the compound III.a, III.b and/or III.c or a mixture of different compounds III.a, III.b and/or III.c

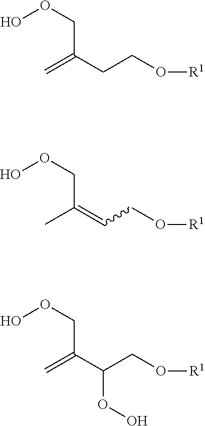

where
in compounds III.a R¹ is hydrogen or —C(═O)R²; and R² is C₁-C₂₀-alkyl;
in compounds III.b R¹ is —C(═O)R²; and R² is C₁-C₂₀-alkyl; and
in compounds III.c R¹ is hydrogen or —C(═O)R²; and R² is C₁-C₂₀-alkyl.
E.67. The hydroperoxide compound according to embodiment E.66, where R² is C₁-C₄-alkyl.
E.68. The hydroperoxide compound according to embodiment E.67, where R² is methyl.
E.69. The hydroperoxide compound according to any of embodiments E.66 to E.68, which is a compound of the formula III.a or III.b or a stereoisomer of the compound of the formula III.a or III.b or a mixture of different stereoisomers of the compound III.a and/or III.b or a mixture of different compounds III.a and/or III.b.
E.70. The hydroperoxide compound according to any of embodiments E.66 to E.68, which is a compound of the formula III.a or III.c or a stereoisomer of the compound of the formula III.a or III.c or a mixture of different stereoisomers of the compound III.a and/or III.c or a mixture of different compounds III.a and/or IIIc.
E.71. The hydroperoxide compound according to any of embodiments E.66 to E.68, which is a compound of the formula III.a or a stereoisomer of the compound of the formula III.a or a mixture of different stereoisomers of the compound III.a or a mixture of different compounds III.a.
E.72. The use of the hydroperoxide compound of the formula III.a, III.b or III.c or of a stereoisomer of the compound of the formula III.a, III.b or III.c or of a mixture of different stereoisomers of the compound III.a, III.b and/or III.c or of a mixture of different compounds III.a, III.b and/or III.c as defined in any of embodiments E.66 to E.71, where however in compound III.b R¹ can also be hydrogen, as intermediates in the synthesis of compounds of the formula I.a or I.b or of a stereoisomer of the compound I.a or I.b or of a mixture of different stereoisomers of the compound I.a and/or I.b or of a mixture of different compounds I.a and/or I.b as defined in any of embodiments E.1 to E.5, or as intermediates in the synthesis of retinol, stereoisomers thereof, derivatives thereof (preferably esters thereof) or stereoisomers of derivatives thereof (preferably stereoisomers of esters thereof).
E.73. The use according to embodiment E.72, of the hydroperoxide compound of the formula III.a or III.b or of a stereoisomer of the compound of the formula III.a or III.b or of a mixture of different stereoisomers of the compound III.a and/or III.b or of a mixture of different compounds III.a and/or III.b.
E.74. The use according to embodiment E.72, of the hydroperoxide compound of the formula III.a or a stereoisomer of the compound of the formula III.a or a mixture of different stereoisomers of the compound III.a or a mixture of different compounds III.a.

Without wishing to be bound by theory, it is assumed that in the method of the invention the conversion of compounds of the formula I.a into compounds I.a and/or I.b pro-cedes via a Schenck ene reaction (=an “ene” reaction in which singlet oxygen is the enophile) and simultaneous (if step (iii) is not carried out) or subsequent (if step (iii) is carried out) transition metal-catalysed dehydration (of the hydroperoxides formed via photooxidation in the Schenck ene reaction).

In the method of the invention the conversion of the compounds of the formula II.a into compounds I.a and/or I.b can be carried out in one step, e.g. as a one-pot-reaction, if the transition metal catalyst is provided in step (i). Step (iii) is of course not carried out in this case. Without wishing to be bound by theory, it is assumed that in the course of step (ii) the compound of the formula II.a is converted into the allylic hydroperoxide which is subsequently dehydrated catalytically to afford compounds I.a and/or I.b. Compounds I.b are assumed to be the result of a double bond isomerization which can occur during the formation of the hydroperoxides and can lead to hydroperoxide intermediates III.b as depicted above or below.

Alternatively, in the method of the invention the conversion of the compounds of the formula II.a into compounds I.a and/or I.b can be carried out in two steps if the transition metal catalyst is not provided in step (i). In this case, step (iii) is mandatory. In the course of step (ii), hydroperoxides are formed which upon contact with the transition metal catalyst are dehydrated to compounds I.a and/or I.b as explained above.

Steps (ii) and (iii) can be repeated several times until the desired conversion rate is obtained. To this purpose, for example, a reaction mixture containing the compound of the formula I.a and the photosensitizer can be circulated in a reactor containing a first reaction zone in which photooxidation takes place and a second reaction zone containing the transition metal catalyst, where dehydration of the hydroperoxides formed in the first reaction zone takes place. This reaction mode can of course also be carried out continuously or semi-continuously by continuously or periodically adding II.a and/or photosensitizer to replace the depleted starting materials and continuously or periodically removing the reaction mixture containing the desired products I.a and/or I.b.

In a preferred embodiment, the invention relates to a method for preparing a compound of the formula I.a or of the formula I.b or a mixture thereof or a stereoisomer of the compound I.a or I.b or a mixture of different stereoisomers of the compound I.a and/or I.b or a mixture of different compounds I.a and/or I.b

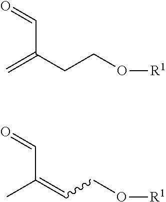

wherein
R¹ is hydrogen or —C(═O)R²; and
R² is C₁-C₂₀-alkyl;
which method comprises
(i) providing a reaction mixture comprising a compound of the formula I.a

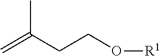

wherein R¹ is as defined above;
a photosensitizer and a transition metal catalyst;
(ii) passing an oxygen-containing gas through the reaction mixture provided in step (i) and simultaneously irradiating the reaction mixture with light;
(iv.1) if desired, after completion of the reaction (to the desired degree), isolating the one or more compounds (I.a) or (I.b) obtained in step (ii); and
(v.1) if desired hydrolysing the one or more compounds (I.a) or (I.b) isolated in step (iv.1) to compounds (I.a) or (I.b) wherein R¹ is hydrogen;
or
(iv.2) if desired, hydrolysing the reaction mixture obtained in step (ii); and
(v.2) if desired, isolating the one or more compounds (I.a) or (I.b) obtained in step (iv.2).

In another preferred embodiment, the invention relates to a method for preparing a compound of the formula I.a or of the formula I.b or a mixture thereof or a stereoisomer of the compound I.a or I.b or a mixture of different stereoisomers of the compound I. and/or I.b or a mixture of different compounds I.a and/or I.b

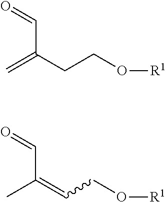

wherein
R¹ is hydrogen or —C(═O)R²; and
R² is C₁-C₂₀-alkyl;
which method comprises
(i) providing a reaction mixture comprising a compound of the formula I.a

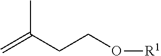

wherein R¹ is as defined above;
and a photosensitize
(ii) passing an oxygen-containing gas through the reaction mixture provided in step (i) and simultaneously irradiating the reaction mixture with light;
(iii) adding a transition metal catalyst to the reaction mixture obtained in step (ii);
(iv.1) if desired, after completion of the reaction (to the desired degree), isolating the one or more compounds (I.a) or (I.b) obtained in step (iii); and
(v.1) if desired hydrolysing the one or more compounds (I.a) or (I.b) isolated in step (iv.1) to compounds (I.a) or (I.b) wherein R¹ is hydrogen;
or
(iv.2) if desired, hydrolysing the reaction mixture obtained in step (iii); and
(v.2) if desired, isolating the one or more compounds (I.a) or (I.b) obtained in step (iv.2).

In yet another preferred embodiment, the invention relates to a method for preparing a compound of the formula I.a or of the formula I.b or a mixture thereof or a stereoisomer of the compound I.a or I.b or a mixture of different stereoisomers of the compound I.a and/or I.b or a mixture of different compounds I.a and/or I.b

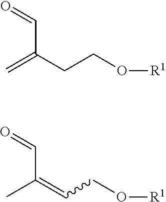

wherein
R¹ is hydrogen or —C(═O)R²; and
R² is C₁-C₂₀-alkyl;
which method comprises
(i) providing a reaction mixture comprising a compound of the formula I.a

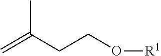

wherein R¹ is as defined above;
and a photosensitize
(ii) passing an oxygen-containing gas through the reaction mixture provided in step (i) and simultaneously irradiating the reaction mixture with light in a first reaction zone;
(iii) passing the reaction mixture obtained in step (ii) through a second reaction zone comprising a transition metal catalyst;
and if desired recirculating the reaction mixture obtained in step (iii) to the first and then to the second reaction zones once or several times;
(iv.1) if desired, after completion of the reaction (to the desired degree), isolating the one or more compounds (I.a) or (I.b) obtained in step (ii); and
(v.1) if desired hydrolysing the one or more compounds (I.a) or (I.b) isolated in step (iv.1) to compounds (I.a) or (I.b) wherein R¹ is hydrogen;
or
(iv.2) if desired, hydrolysing the reaction mixture obtained in step (iii); and
(v.2) if desired, isolating the one or more compounds (I.a) or (I.b) obtained in step (iv.2).

In compounds I.a, I.b and II.a, R² is preferably C₁-C₄-alkyl or n-C₁₅-alkyl, more preferably C₁-C₄-alkyl, even more preferably C₁-C₂-alkyl and in particular methyl.

R¹ is preferably —C(═O)R², especially in compounds II.a.

The transition metal catalyst used in step (i) or (iii) can be a homogeneous or a heterogeneous catalyst. In homogeneous catalysts, the catalyst is in the same phase as the reactants or products, whereas in heterogeneous catalysis, the phase of the catalyst differs from that of the reactants or products. A heterogeneous catalyst in terms of the present invention is thus a catalyst which is not soluble in the reaction medium.

The transition metal catalyst used in step (i) or (iii) is a preferably a heterogeneous catalyst.

Heterogeneous catalysts are generally either full catalysts or supported catalysts. A full catalyst is a catalyst in which the active metal in its elementary or oxidised form makes up the major part, i.e. more than 50% by weight, in particular at least 80% by weight of the catalyst in its active form. A supported catalyst is a catalyst where the active metal is supported on a support material.

Preferably, the heterogeneous transition metal catalyst is a supported catalyst.

In metal salts, the catalytically active metal can be (part of) the cation part or part of the anion.

Metal compounds in this context are for example metal oxides (which are generally not rated among metal salts) and metal complexes (coordination compounds).

The active metal of the metal catalyst is a transition metal. “Active” metal means that this metal is the catalytically active site. The transition metal catalyst may contain other metals, e.g. for the purpose of charge balance if the active metal is part of an anion (like in permanganates, chromates, dichromates and the like), which are not necessarily transition metals.

Preferably, the transition metal catalyst comprises a transition metal of group 4 to 12 of the Periodic Table of Elements in elemental or oxidized form. More preferably, the transition metal catalyst comprises a transition metal of group 4 to 12 of the Periodic Table of Elements in oxidized form.

The group numbering relates to the IUPAC nomenclature of 1985. Groups 4 to 12 are thus the Ti, V, Cr, Mn, Fe, Co, Ni, Cu and Zn groups.

Preferably, the transition metal catalyst comprises a transition metal of period 4 of group 4 to 12 (i.e. Ti, V, Cr, Mn, Fe, Co, Ni, Cu, Zn) of the Periodic Table of Elements in elemental or oxidized form. More preferably, the transition metal catalyst comprises a transition metal of period 4 of group 4 to 12 (i.e. Ti, V, Cr, Mn, Fe, Co, Ni, Cu, Zn) of the Periodic Table of Elements in oxidized form.

Preferably, the transition metal catalyst comprises a transition metal salt or a transition metal oxide, more preferably a salt or an oxide of a metal of group 4 to 12 of the Periodic Table of Elements, and even more preferably a salt or an oxide of a metal of period 4 of group 4 to 12 of the Periodic Table of Elements.

The metal salt or oxide is preferably a transition metal C₂-C₈-carboxylate (R—C(═O)O⁻, where R is C₁-C₇-alkyl, e.g. acetate, propionate, butyrate and the like), halide (e.g. fluoride, chloride, bromide, iodide), nitrate, phosphate, sulfate, chlorate, perchlorate or oxide, more preferably a C₂-C₈-carboxylate, halide, nitrate, phosphate, sulfate, chlorate, perchlorate or oxide of a transition metal of group 4 to 12 of the Periodic Table of Elements, even more preferably a C₂-C₈-carboxylate, halide, nitrate, phosphate, sulfate, chlorate, perchlorate or oxide of a transition metal of the 4^{th} period of group 4 to 12 of the Periodic Table of Elements; or is a chromate, chlorochromate, dichromate or permanganate.

More preferably, the salt or oxide of the transition metal is a C₂-C₈-carboxylate, halide, nitrate, phosphate, sulfate, chlorate, perchlorate, or oxide of Cu, Cr, Fe or Ni, or is a chromate or dichromate, the chromate or dichromate being preferably an alkali metal, Zn or Fe chromate or dichromate.

Even more preferably, the salt or oxide of the transition metal is selected from the group consisting of Cu(II) acetate, Cu(ClO₄)₂, CuCl₂, CuO, Co(II) acetate, CrO₃ and an alkali metal dichromate, the latter being preferably K₂Cr₂O₇.

In particular, the transition metal catalyst comprises Cr(VI) oxide (CrO₃).

As said, the transition metal catalyst is preferably a supported catalyst.

Suitable support materials are known in the art and are for example carbon, such as activated carbon, alumina, silica, silicon carbide, alumosilicates, such as zeolites, titanium dioxide, zirconium dioxide, or organic polymers.

Suitable organic polymers are for example vinylpyridine homo- and copolymers, N-vinylpyrrolidine homo- and copolymers, styrene homo- and copolymers, polyurethanes, acrylate homo- and copolymers and methacrylate homo- and copolymers.

Preferably, the support material is an organic polymer. The organic polymer is preferably selected from the group consisting of vinylpyridine homo- and copolymers, N-vinylpyrrolidine homo- and copolymers, styrene homo- and copolymers, polyurethanes, acrylate homo- and copolymers and methacrylate homo- and copolymers, more preferably from 2-vinylpyridine homo- and copolymers, 4-vinylpyridine homo- and copolymers and N-vinylpyrrolidine homo- and copolymers, even more preferably from poly(2-vinylpyridine), poly(4-vinylpyridine), copolymers, preferably block copolymers, of 2-vinylpyridine and methyl methacrylate; and poly(N-vinylpyrrolidine), and in particular from polyvinylpyridines, specifically poly(2-vinylpyridine) or poly(4-vinylpyridine), very specifically poly(4-vinylpyridine).

The molecular weights of the polymers can vary within wide ranges. For example, the polymers can have number average molecular weights (Mₙ) of from 500 to 1,000,000, e.g. from 2,000 to 500,000 or from 10,000 to 300,000, and weight average molecular weights (M_{w}) of from 700 to 2,000,000, e.g. from 5000 to 1,000,000 or from 15,000 to 500,000.

To enhance stability and/or loading capacity, the polymers can moreover be cross-linked.

A typical crosslinking material for polyvinylpyridine is for example divinylbenzene. The degree of crosslinking depends on the desired rigidity of the material and can range from 1 to 40%, preferably from 2 to 30%, more preferably from 10 to 30%, in particular from 20 to 30%. The degree of crosslinking is the quotient of the number of moles of crosslinker and the total number of moles of building blocks present in the crosslinked macromolecular network, here expressed in % (e.g. a polyvinylpyridine crosslinked with 1% of divinylbenzene means to 99 mol of vinylpyridine crosslinked with 1 mol of divinylbenzene). The degree of crosslinking is generally determined analytically, e.g. rheologically.

Depending on the metal species of the catalyst and on the nature of the support material, the bonding between catalytic metal species and support material can be, for example, by adsorption, electrostatic interaction, coordinative bonding or covalent bonding. Just by way of example, the preferably used polyvinylpyridine support material can interact electrostatically with negatively loaded metal species (i.e. if the metal is part of an anion, such as in permanganates, chromates or dichromates) if the nitrogen atom of the pyridine rings is quaternized, e.g. by protonation or alkylation. If the metal species in a metal oxide or a metal salt in which the metal is the cation or part of a complex cation or in which the metal is part of an anion and the counter cation is one to which the nitrogen atom of the pyridine can coordinate (such as in Ag, Fe or Zn chromate or dichromate), the polyvinylpyridine support material generally interacts coordinatively, the nitrogen atom of the pyridine rings acting as a ligand.

The catalyst loading of the active metal (i.e. of the transition metal, e.g. of the group 4-12 transition metal), i.e. the amount of active metal in the supported catalyst, is preferably in the range of from 1 to 20% by weight, more preferably from 1 to 15% by weight, even more preferably from 1 to 10% by weight, in particular from 2 to 10% by weight, relative to the total (dry) weight of the supported catalyst. The percentages refer to the active metal only, and not to any salt or oxide or other form in which the metal is actually present (e.g. in case of Cr being present as CrO₃ or chromate or dichromate etc., the percentages relate to Cr only). The loading can be determined analytically, for example by atomic absorption spectrometry, or can be calculated from the preparation method.

Alternatively, the catalyst loading of the active metal salt or oxide or complex (i.e. of the salt or oxide or complex of the transition metal, e.g. the group 4-12 transition metal), i.e. the amount of the form in which the active metal is present in the supported catalyst, is preferably in the range of from 2 to 40% by weight, more preferably from 3 to 35% by weight, even more preferably from 5 to 30% by weight, in particular from 5 to 20% by weight, relative to the total (dry) weight of the supported catalyst. The percentages refer to the weight of the form in which active metal is present, i.e. to the salt or oxide or other form in which the metal is actually present (e.g. in case of Cr being present as CrO₃ or chromate or dichromate etc., the percentages relate to the CrO₃ or chromate or dichromate). The loading can be determined analytically, for example by atomic absorption spectrometry, or can be calculated from the preparation method.

Supported transition metal catalysts suitable for the method according to the present invention are either commercially available or can be obtained by methods known in the art, e.g. by bringing the metal species or a precursor thereof and the support material or a precursor thereof into intimate contact with each other, where necessary in the presence of a solvent which can subsequently be removed; where necessary or expedient under heating. Where necessary, the precursor of the metal species or of the support material is first treated to convert it into that form which can interact with the partner. Just by way of example, if the support material is polyvinylpyridine which is to interact electrostatically with a negatively loaded metal species, polyvinylpyridine is first quaternized, e.g. by bringing it into contact with a Bronsted acid to protonate (a part of) the nitrogen atoms of the pyridine ring or with an alkylation agent (e.g. an alkyl bromide or iodide or sulfate, e.g. butylbromide) to alkylate (a part of) the nitrogen atoms of the pyridine ring, and the resulting support material with quaternized nitrogen atoms of the pyridine ring is then brought into contact with a salt of the negatively loaded metal species to exchange the anions and thus bind the metal species via ionic interaction to the support material.

The catalyst (calculated on the basis of the active metal content) is preferably used in an amount of from 0.0001 to 15 mol-%, more preferably from 0.001 to 10 mol-%, even more preferably from 0.01 to 5 mol-%, in particular from 0.1 to 5 mol-%, specifically from 1 to 5 mol-%, more specifically from 1 to 3 mol-%, relative to 1 mol of the compound II.a.

The support material is commercially available (e.g. from Acros Chemicals, Merck or Vertellus). The polymeric support material is often commercialized as ionic exchange resin.

The heterogeneous catalyst can be used in bulk, loose form or can be immobilized, e.g. in a fixed bed.

As explained above, the photosensitizer used in the method of the invention is a compound which, when subjected to irradiation (generally to electromagnetic radiation in the UV, in the visible or in the near IR region) can convert triplet oxygen to singlet oxygen: Upon irradiation, the sensitizer forms the corresponding excited singlet state. Intersystem crossing affords the excited triplet state of the sensitizer, thus transferring energy to triplet oxygen to form singlet oxygen. Singlet oxygen is the species which oxidizes the compounds II.a to the respective hydroperoxides (or double bond isomers thereof).

Preferably, the photosensitizer can convert triplet oxygen to singlet oxygen when subjected to electromagnetic radiation in the near UV, in the visible or in the near IR region, more preferably in the visible or in the near IR region, in particular in the visible region.

Preferably, the photosensitizer is selected from the group consisting of fluorescein, eosin, rose Bengal (RB), erythrosine, tetraphenylporphyrin (to be more precise 5,10,15,20-tetraphenyl-21H,23H-porphine; TPP; 2HTPP), cobalt-tetraphenylporphyrin (Co-TPP; i.e. the cobalt complex of TPP with Co(II)), zinc-tetraphenylporphyrin (Zn-TPP; i.e. the zinc complex of TPP with Zn(II)), hematoporphyrin, rhodamine B, basacryl brilliant red, methyl violet, methylene blue, fullerene C₆₀, fullerene C₇₀, graphene, carbon nanotubes, Ru(bpy)₃²⁺ salts (bpy=2,2′-bipyridine) (e.g. tris(2,2′-bipyridine)ruthenium(II) hexafluorophosphate, tris(2,2′-bipyridine)ruthenium(II) chloride, often as hexahydrate) Ru(phen)₃²⁺ salts (phen=1,10-phenanthroline) (e.g. dichloro-tris(1,10-phenanthroline)ruthenium(II) chloride), cercosporin, hypocrellin-A and mixtures thereof. More preferably, the photosensitizer is selected from the group consisting of tetraphenylporphyrin, cobalt-tetraphenylporphyrin, zinc-tetraphenylporphyrin, methylene blue, Ru(bpy)₃²⁺ salts (in particular tris(2,2′-bipyridine)ruthenium(II) hexafluorophosphate, tris(2,2′-bipyridine)ruthenium(II) chloride or its hexahydrate) and Ru(phen)₃²⁺ salts (in particular dichlorotris(1,10-phenanthroline)ruthenium(II) chloride), in particular from tetraphenylporphyrin, zinc-tetraphenylporphyrin and Ru(bpy)₃²⁺ salts (in particular tris(2,2′-bipyridine)ruthenium(II) hexafluorophosphate, tris(2,2′-bipyridine)ruthenium(II) chloride or its hexahydrate); and is specifically tetraphenylporphyrin.

The photosensitizer can be provided in step (i) in very low amounts. However, during irradiation, a part of the photosensitizer may degrade, leading to decreasing generation of singlet oxygen and thus slowing down the conversion of the compounds II.a to the corresponding hydroperoxides and eventually to the desired compounds I.a and/or I.b. Thus, either higher amounts of the photosensitizer are provided in step (i) or in the course of step (ii) further photosensitizer is added if this is depleted during irradiation.

The degree of depletion/degradation of the photosensitizer can be monitored in the course of step (ii), e.g. by UV/Vis spectroscopy, which can also be carried out in line.

The photosensitizer is preferably used in an overall amount of from 0.00001 to 1 mol-%, relative to 1 mol of the compound of the formula I.a. Overall amount means the total amount of photosensitizer provided in step (i) and added in the course of step (ii), if applicable. More preferably, the photosensitizer is used in an overall amount of from 0.0001 to 0.5 mol-%, even more preferably 0.0001 to 0.2 mol-%, e.g. 0.0005 to 0.2 mol-% or 0.001 to 0.1 mol-%, relative to 1 mol of the compound of the formula II.a.

Alternatively, the photosensitizer is preferably used in an overall amount of from 0.0000001 to 0.01 mol, more preferably from 0.000001 to 0.01 mol or from 0.000001 to 0.005 mol, even more preferably from 0.000001 to 0.002 mol or from 0.000005 to 0.01 mol, particularly preferably from 0.00001 to 0.001 mol, specifically from 0.00001 to 0.005 mol or from 0.00001 to 0.001 mol, e.g. from 0.00001 to 0.0005 mol, per 1 mol of the compound of the formula II.a.

Typical photosensitizers are dyes and are thus excitable with electromagnetic radiation in the near UV, visible or near infrared (near IR; NIR) electromagnetic spectrum. Thus, preferably in step (ii) the reaction mixture is irradiated with light in the near UV, visible or near IR range. More preferably, in step (ii) the reaction mixture is irradiated with light in the visible or near IR range, and in particular in the visible range.

Preferably, in step (ii) the reaction mixture is irradiated with light in the wavelength range of from 350 to 800 nm, more preferably from 350 to 680 nm, even more preferably in the wavelength range of from 400 to 650 nm, even more preferably from 400 to 580 nm, and in particular from 400 to 500 nm. The optimum wavelength range depends i.a. on the photosensitizer used and can for example be determined by short tests, if not anyway known to the skilled person, or can be selected by means of UV spectroscopy.

For instance, if the photosensitizer is tetraphenylporphyrin, cobalt-tetraphenylporphyrin or zinc-tetraphenylporphyrin, in step (ii) the reaction mixture can for example be irradiated with light in the wavelength range of from 400 to 430 nm, preferably 400 to 420 nm, e.g. 400 to 410 nm; if the photosensitizer is methylene blue, in step (ii) the reaction mixture can for example be irradiated with light in the wavelength range of from 600 to 620 nm, and if the photosensitizer is a Ru(bpy)₃²⁺ salt or a Ru(phen)₃²⁺ salt, in step (ii) the reaction mixture can for example be irradiated with light in the wavelength range of from 450 to 480 nm, preferably from 460 to 475 nm.

In step (ii) the reaction mixture is preferably irradiated with monochromatic light.

In theory, monochromatic light is light with a single constant frequency/light of a single vacuum wavelength range. In practice, however, no radiation can be totally monochromatic. Thus, in practice, “monochromatic” light—even from lasers or spectral lines—always consists of components with a range of frequencies of non-zero width. In terms of the present invention, monochromatic light is understood as light produced by state-of-the-art sources of monochromatic light, such as monochromators, optical filters, Hg vapour lamps (high, middle or low pressure lamps), generally in combination with an optical filter, doped Hg vapour lamps, if necessary in combination with an optical filter, Na vapour lamps (high or low pressure lamps), lasers, or, in particular, monochromatic LEDs.

Preferably, irradiation in step (ii) is carried out using a monochromatic light source, preferably an electroluminescent lighting device emitting monochromatic light, where at least 90% of the light emitted by said monochromatic light source is in the wavelength range of from 350 to 800 nm, preferably from 350 to 680 nm, more preferably in the wavelength range of from 400 to 650 nm, even more preferably from 400 to 580 nm, in particular from 400 to 500 nm.

Preferably, irradiation in step (ii) is carried out using an electroluminescent lighting device emitting monochromatic light, where the electroluminescent lighting device consists of at least one LED.

The oxygen-containing gas used in step (ii) is preferably selected from the group consisting of oxygen, air and mixtures of oxygen and nitrogen containing oxygen in a range of from 1 to 99% by weight, relative to the total weight of the mixture. More preferably, the oxygen-containing gas used in step (ii) is selected from the group consisting of oxygen, air and mixtures of oxygen and nitrogen containing oxygen in a range of from 20 to 99% by weight, and is in particular oxygen.

“Passing an oxygen-containing gas through the reaction mixture provided in step (i)” is not limited to bubbling an oxygen-containing gas through said mixture, thus letting a substantial part of the oxygen-containing gas escape, but also encompasses inserting into and keeping an oxygen-containing gas in the reaction mixture, e.g. by using a closed, generally pressurized reaction vessel.

Preferably, steps (ii) and (iii) are carried out neat (i.e. in substance). “Neat” or “in substance” means that no additional solvent is present. The specification “additional” takes account of the fact that the starting compound II.a and also the intermediately formed hydroperoxides can serve as solvent or dispersant for the photosensitizer and the transition metal catalyst. To carry out step (ii) neat, the reaction mixture provided in step (i) is for example either prepared by mixing the starting materials (compounds II.a, photosensitizer, transition metal catalyst if the reaction is to be carried out in one step) in the absence of any additional solvent, or by mixing the starting materials in the presence of an additional solvent and then removing the same before step (ii) is carried out.

In an alternatively preferred embodiment, steps (ii) and (iii) are carried out in the presence of a chlorinated C₁-C₂-alkane. To this purpose, said chlorinated C₁-C₂-alkane is expediently provided in the reaction mixture of step (i). The molar ratio of the compound (II.a) provided in step (i) to the chlorinated C₁-C₂-alkane is of from 50:1 to 1:1.5, more preferably from 30:1 to 1:1, even more preferably from 20:1 to 1:1, in particular from 15:1 to 2:1. The chlorinated C₁-C₂-alkane is preferably trichloromethane or tetrachloromethane.

More preferably, however, steps (ii) and (iii) are carried out neat.

Step (ii) is preferably carried out at a temperature of from −20 to 150 °C, more preferably from 0 to 70 °C, e.g. at from 0 to 60 °C or from 5 to 50 °C or from 20 to 50 °C.

Step (ii) is preferably carried out at a pressure of from atmospheric pressure to 100 bar (10 MPa). Atmospheric pressure means the local ambient pressure, and thus roughly 1013.25 hPa±200 hPa. In a more preferred embodiment, step (ii) is carried out at a pressure of from atmospheric pressure to 10 bar (>0.1 to 1 mPa).

In step (ii) either the complete reaction mixture or only a distinct portion of the reaction mixture is irradiated. The latter occurs for example if only a portion of the reaction mixture (e.g. only 20 to 90% or 30 to 80% or 50 to 80% by weight of the reaction mixture) is passed by the irradiation source.

In a specific embodiment, step (ii) is carried out thus that a part of the starting compound II.a remains unreacted. This ensures that compound II.a can still serve as solvent for the other substances present in the reaction mixture. Preferably, at least 20%, more preferably at least 50% of the initially charged amounts of compound I.a remain unreacted in step (ii). After work-up, isolation and if desired purification, compound II.a can be reused in step (i). The degree of conversion of compounds II.a can be determined via usual means, such as periodical or continuous sample collection and analysis or in-line analysis of the composition of the reaction mixture, or by passing oxygen through the reaction mixture in predetermined substoichiometric amounts. Reaction in step (ii) is for example interrupted by ceasing the oxygen feed and/or by ceasing irradiation.

Steps (ii) and (iii) can be carried out in any reactor known in the art as suitable for photooxidations. Suitable reactors contain at least a means for introducing the oxygen-containing gas and a radiation source. Moreover, the reactor expediently contains a stirrer and means for cooling or heating.

Examples for suitable reactors are side-loop photoreactors, continuous flow-photoreactors or submersible photoreactors.

For circulating the reaction mixture containing the compound of the formula II.a and the photosensitizer in a reactor containing a first reaction zone in which photooxidation takes place and a second reaction zone containing the transition metal catalyst, where dehydration of the hydroperoxides formed in the first reaction zone takes place, a suitable reactor contains expediently pumps and lines to circulate the reaction mixture, i.e. from one zone to the other. For instance, a photoreactor is connected to a reactor containing the transition metal catalyst thusly that the reaction mixture can be circulated between photoreactor and metal transition catalyst-containing reactor.

After completion of the reaction, the reaction mixture obtained in step (ii) (if step (iii) is not carried out) or step (iii) is generally worked up. “Completion” of the reaction in this context does not mandatorily mean maximum conversion of the starting material, but conversion to a desired degree. As explained above, especially if step (ii) is carried out neat, the starting compound II.a generally serves as solvent. In this case, it is expedient to stop the reaction distinctly before maximum conversion of II.a.

Work-up of the reaction mixture obtained in step (ii) (if step (iii) is not carried out) or step (iii) can be carried out by usual means, such as separation from the transition metal catalyst, if necessary, and isolation of the desired reaction products I.a and/or I.b (step (iv.1) by separation from the further components of the reaction mixture, such as unreacted compound II.a, photosensitizer or undesired side products and, if desired, separation from each other. Separation can be carried out by usual means, such as extractive, distillative or chromatographic methods.

If compounds I.a or I.b are formed as different stereoisomers, these can be separated from each other if desired.

To obtain compounds I.a and/or I.b wherein R¹ is hydrogen, either the compounds I.a and/or I.b isolated according to step (iv.1) or the reaction mixture as obtained from step (ii) (if step (iii) is not carried out) or from step (iii) is hydrolized, e.g. by reaction with an acid or with a base. Hydrolysis is of course necessary (to obtain compounds I.a and/or I.b wherein R¹ is hydrogen) if in compounds II.a (and thus also in the resulting compounds I.a and/or I.b) R¹ is —C(O)R², but might also be necessary if in in compounds II.a R¹ is hydrogen, since a part of the hydroxyl group might be acylated by the acylating agent, especially if this is used in excess.

If the reaction mixture as obtained from step (ii) (if step (iii) is not carried out) or from step (iii) is hydrolized (step (v.1)), the obtained reaction mixture can be worked-up and the desired reaction products I.a and/or I.b (step (iv.1) can be separated from the further components of the reaction mixture and also from each other by usual means, such as extraction, distillation or chromatographic methods.

The process of the invention offers a simple method for the preparation of compounds I.a and/or I.b starting from the readily available bulk chemicals isoprenol and isoprenol esters II.a. Compounds I.a and I.b can serve as intermediates in the preparation of retinol, stereoisomers thereof, derivatives thereof (preferably esters thereof; in particular esters in which the OH group of retinol is esterified to —O—C(O)R²) or stereoisomers of derivatives thereof (preferably stereoisomers of esters thereof; in particular of esters in which the OH group of retinol is esterified to —O—C(O)R²). Compounds I.a can be converted into compounds I.b by a known Pd-catalyzed double-bond isomerization reaction, as described e.g. in U.S. Pat. No. 4,124,619 or CN 103467287.

The invention relates moreover to the use of the compound of the formula I.a or I.b different from (E)-4-acetoxy-2-methylbut-2-enal or of a stereoisomer of the compound I.a or I.b different from (E)-4-acetoxy-2-methylbut-2-enal or of a mixture of different stereoisomers of the compound I.a and/or I.b or of a mixture of different compounds I.a and/or I.b as defined above as intermediates in the synthesis of retinol, stereoisomers thereof, derivatives thereof (preferably esters thereof; in particular esters in which the OH group of retinol is esterified to —O—C(O)R²) or stereoisomers of derivatives thereof (preferably stereoisomers of esters thereof; in particular esters in which the OH group of retinol is esterified to —O—C(O)R²). As explained above, compounds I.a can be readily converted into I.b. The latter can be reacted in a Wittig reaction with a β-ionylidenethyltriphenylphosphonium salt to retinol (this results directly if in I.b R¹═H), stereoisomers thereof, esters thereof (esters in which the OH group of retinol is esterified to —O—C(O)R² result directly if in I.b R¹=—C(O)R²) or stereoisomers of esters thereof, as described for example by J. Paust et al. in Carotenoids, Birkhsuser, 1996, vol. 2, pp. 258-292, in U.S. Pat. No. 5,087,762 or by H. Ernst, Pure Appl. Chem. 2002, 74, 2213 or by G. L Parker et al. in Tetrahedron 2016, 72, 1645-1652. Other retinol derivatives are obtainable by usual means; e.g. by esterification of retinol (or stereoisomers thereof) with acids or acid derivatives different from R²—C(O)OH or derivatives thereof; or by oxidation of retinol (or stereoisomers thereof) to retinal (or stereoisomers thereof) or retinoic acid (or stereoisomers thereof). Retinol (or stereoisomers thereof) be obtained by saponification (ester cleavage) of retinol esters (or stereoisomers thereof). These conversions are well known in the art.

The invention relates furthermore to a hydroperoxide compound of the formula III.a, III.b or III.c or a stereoisomer of the compound of the formula III.a, III.b or III.c or a mixture of different stereoisomers of the compound III.a, and/or III.b and/or III.c or a mixture of different compounds III.a, III.b and/or III.c

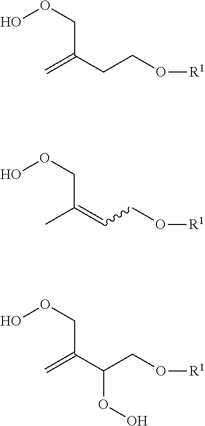

where
in compounds III.a R¹ is hydrogen or —C(═O)R²; and R² is C₁-C₂₀-alkyl;
in compounds III.b R¹ is —C(═O)R²; and R² is C₁-C₂₀-alkyl; and
in compounds III.c R¹ is hydrogen or —C(═O)R²; and R² is C₁-C₂₀-alkyl.

The hydroperoxides III.a, III.b and III.c are formed in step (ii) of the method of the invention. If the reaction mixture provided in step (i) does not contain an acylation agent, the hydroperoxides formed in step (ii) can be detected and also isolated, since in the absence of acylation agents their further reaction/decomposition is rather slow.

The invention relates preferably to a hydroperoxide compound of the formula III.a or III.b or a stereoisomer of the compound of the formula III.a or III.b or a mixture of different stereoisomers of the compound III.a and/or III.b or a mixture of different compounds III.a and/or III.b.

The invention relates alternatively preferably to a hydroperoxide compound of the formula III.a or III.c or a stereoisomer of the compound of the formula III.a or III.c or a mixture of different stereoisomers of the compound III.a and/or III.c or a mixture of different compounds III.a and/or III.c.

The invention relates in particular to a hydroperoxide compound of the formula III.a or a stereoisomer of the compound of the formula III.a or a mixture of different stereoisomers of the compound III.a or a mixture of different compounds III.a.

The invention relates furthermore to the use of the hydroperoxide compound of the formula III.a, III.b or III.c or of a stereoisomer of the compound of the formula III.a, III.b or III.c or of a mixture of different stereoisomers of the compound III.a, III.b and/or III.c or of a mixture of different compounds III.a, III.b and/or III.c as defined above, where however in compound III.b R¹ can also be hydrogen, as intermediates in the synthesis of compounds of the formula I.a or I.b or of a stereoisomer of the compound I.a or I.b or of a mixture of different stereoisomers of the compound I.a and/or I.b or of a mixture of different compounds I.a and/or I.b as defined above, or as intermediates in the synthesis of retinol, stereoisomers thereof, derivatives thereof (preferably esters thereof; in particular esters in which the OH group of retinol is esterified to —O—C(O)R²) or stereoisomers of derivatives thereof (preferably stereoisomers of esters thereof; in particular esters in which the OH group of retinol is esterified to —O—C(O)R²).

The conversion of the compounds III.a and/or III.b into compounds I.a and/or I.b is carried out by subjecting compounds III.a and/or III.b to step (iii) and optionally steps (iv) and (v) of the method of the invention described above. Compounds I.a and/or I.b can be converted into retinol, stereoisomers thereof, derivatives thereof or stereoisomers of derivatives thereof as described above.

The invention relates preferably to the use of the hydroperoxide compound of the formula III.a or III.b or of a stereoisomer of the compound of the formula III.a or III.b or of a mixture of different stereoisomers of the compound III.a and/or III.b or of a mixture of different compounds III.a and/or III.b as defined above, where however in compound III.b R¹ can also be hydrogen, as intermediates in the synthesis of compounds of the formula I.a or I.b or of a stereoisomer of the compound I.a or I.b or of a mixture of different stereoisomers of the compound I.a and/or I.b or of a mixture of different compounds I.a and/or I.b as defined above, or as intermediates in the synthesis of retinol, stereoisomers thereof, derivatives thereof (preferably esters thereof; in particular esters in which the OH group of retinol is esterified to —O—C(O)R²) or stereoisomers of derivatives thereof (preferably stereoisomers of esters thereof; in particular esters in which the OH group of retinol is esterified to —O—C(O)R²).

The invention relates in particular to the use of a hydroperoxide compound of the formula III.a or a stereoisomer of the compound of the formula III.a or a mixture of different stereoisomers of the compound III.a or a mixture of different compounds III.a as intermediate in the synthesis of compounds of the formula I.a or I.b or of a stereoisomer of the compound I.a or I.b or of a mixture of different stereoisomers of the compound I.a and/or I.b or of a mixture of different compounds I.a and/or I.b as defined above, or as intermediates in the synthesis of retinol, stereoisomers thereof, derivatives thereof (preferably esters thereof; in particular esters in which the OH group of retinol is esterified to —O—C(O)R²) or stereoisomers of derivatives thereof (preferably stereoisomers of esters thereof; in particular esters in which the OH group of retinol is esterified to —O—C(O)R²).

The following examples serve as further illustration of the invention.

### EXAMPLES

The compounds were characterized by ¹H-NMR and partially also by ¹³C-NMR. The NMR analysis was carried out on a Bruker 400 MHz and 500 MHz Spectrometer in CDCl₃. The signals are characterized by chemical shift (ppm) vs. tetramethylsilane, by their multiplicity and by their integral (relative number of hydrogen atoms given). The following abbreviations are used to characterize the multiplicity of the signals: m=multiplett, q=quartett, t=triplett, d=doublet and s=singlett.

A. Preparation of the Metal Catalyst CrO₃—PVPy (CrO₃ on poly-(4-vinylpyridine))

### Apparatus:

Four-neck reaction flask, stirrer, thermometer, dropping funnel, nitrogen blanketing.

### General Proceeding:

In a 1 L reaction flask, under a nitrogen atmosphere, 200 g of poly-(4-vinylpyridine, 25% cross-linked with divinylbenzene from Acros Chemicals (sieved to grain size 0.85-1.0 mm) were placed in 400 mL of water. At room temperature, under slight cooling, a solution of 20.0 g (0.2 mmol) chromium(VI) oxide and 60 mL water was added drop-wise. The orange reaction mixture was stirred overnight at room temperature at 150 rpm and then filtered through a glass suction filter. The spherical residue was washed twice with 500 mL of water each and then dried for at least 12 hours in a vacuum drying oven at 50 °C and 30 mbar. The chromium content was analyzed by atomic absorption spectrometry. CrO₃—PVPy catalysts with higher or lower amounts of CrO₃ can be obtained by using correspondingly adapted amounts of CrO₃ and/or polymer.

### B. Photooxidation

### 1. Photooxidation of Isoprenyl Acetate in a G1-Corning Photoreactor and Rearrangement/Dehydration Using CrO₃—PVPy

### Examples 1 to 3

### Apparatus:

G1-Corning photoreactor (5 tempered G1 plates, layer thickness approx. 1 mm, each irradiated on both sides by LEDs, total of 200 LEDs with a wavelength of 405 nm, total radiometric power 195 W);
100 mL miniplant reactor with impeller stirrer as feed vessel;
overflow valve 8 bar;
glass column (d=2.5 cm) with CrO₃—PVPy filling;
two FLOM double-stroke piston pumps connected in parallel, each max. 99.9 mL/min

### Experiment:

266.4 g (2078.6 mmol, 1 eq) of isoprenyl acetate (IPA) were placed in a glass vial, 11.71 g (60.3 mmol, 0.029 eq) of dimethyl phthalate were added as an internal NMR standard, and 43.2 mg (70.27 μmol, 0.000034 eq) of the photosensitizer tetraphenyl porphyrin (TPP) were dissolved in the mixture. The reaction mixture was transferred to the Corning photoreactor via the 100 mL miniplant reactor, stirred at 100 rpm and circulated in the system by pumping the solution downstream of the Corning photoreactor through a relief valve, which opened only at 8 bar to increase oxygen solubility. Downstream of the valve, the reaction mixture passes unpressurized through a glass column containing 38 g of CrO₃—PVPy (loading approx. 15% by weight of CrO₃, relative to the total weight of the supported catalyst), where the hydroperoxide formed in the Corning photoreactor was converted to compounds I.a and I.b wherein R¹═C(O)CH₃. From there, the reaction mixture was pumped back into the miniplant reactor. At the temperature indicated in Table 1, the solution was circulated between miniplant reactor, Corning photoreactor and CrO₃—PVPy glass column and irradiated for a period of 6 hours while 1.5 L/h of oxygen were introduced into the Corning photoreactor. During the reaction, up to 69 mg (112 μmol) of TPP were replenished in portions. At the end of the experiment, the reaction mixture was analysed without further workup. The results are summarized in Table 1.

TABLE 1

| | | Conversion | Yield I.a^{#} | | Yield I.b^{##} | |
|---|---|---|---|---|---|---|
| No. | T [°C] | IPA [%] | [g] | [%]¹ | [g] | [%]¹ |
| 1 | 20 | 10.2 | 17.2 | 5.8 | 6.1 | 2.0 |
| 2 | 40 | 16.7 | 26.7 | 9.1 | 10.2 | 3.5 |
| 3 | 50 | 17.9 | 25.2 | 8.5 | 9.7 | 3.3 |

¹Yield relative to the amount of IPA as used in the reaction (here 2078.6 mmol)
^{#}Compound I.a wherein R¹ = C(O)CH₃
^{##}Mixture of E and Z isomers of compound I.b wherein R¹ = C(O)CH₃
I.a^{#}: ¹H-NMR (400 MHz, CDCl₃): δ = 9.56 (1H), 6.11 (1H), 6.36 (1H), 4.19 (2H), 2.61 (2H), 2.03 (3H)
I.b^{##}: ¹H-NMR (400 MHz, CDCl₃): δ = 9.56 (1H), 6.10 (1H), 4.60 (2H), 2.03 (3H), 1.84 (3H)

### 2. Photooxidation of Isoprenyl Acetate in a Ring Gap Reactor and Simultaneous Rearrangement/Dehydration Using CrO₃—PVPy

### Example 4

### Apparatus:

Temperature-controlled annular gap reactor, volume approx. 500 mL, diameter 40 mm, with displacer 20 mm, layer thickness 10 mm; irradiated from outside to inside;
LED lamp (2 half-shells with a total of 256 LEDs with a wavelength of 420 nm, total radiometric power 86 W at 4.2 A);
Harvard syringe pump for replenishing the photosensitizer (dissolved in reaction medium),
Glass column (d=3.5 cm) with CrO₃—PVPy filling,
Ismatec® gear pump, pump head max. 540 mL/min.

### Experiment:

403.8 g (3151 mmol, 1 eq) of isoprenyl acetate (IPA) were placed in a glass bottle, 17.75 g (91.4 mmol, 0.029 eq) of dimethyl phthalate were added as an internal NMR standard and 4.0 mg (6.5 μmol, 0.0000021 eq) of the photosensitizer tetraphenylporphyrin (TPP) were dissolved in the mixture. The reaction mixture was introduced into the apparatus via a dropping funnel and circulated between the irradiated annular gap reactor and the glass column containing 73 g of CrO₃—PVPy (loading about 14% by weight of CrO₃, relative to the total weight of the supported catalyst), where the hydroperoxide formed was converted to compounds I.a and I.b wherein R¹═C(O)CH₃. At 40 °C, the solution was irradiated at 420 nm for a period of 5 hours, while 2 L/h of oxygen were introduced into the annular gap reactor from below via a frit. During the reaction, 24 mg (39 μmol) of tetraphenylporphyrin dissolved in reaction medium was continuously added via a syringe pump, depending on the transmission. At the end of the experiment, the reaction mixture was analysed without further workup. The results are summarized in Table 2.

TABLE 2

| | Conversion | Yield I.a^{#} | | Yield I.b^{##} | |
|---|---|---|---|---|---|
| No. | IPA [%] | [g] | [%]¹ | [g] | [%]¹ |
| 4 | 12.8 | 28.9 | 6.4 | 11.3 | 2.5 |

¹Yield relative to the amount of IPA as used in the reaction (here 3151 mmol)

### 3. Photooxidation of Isoprenyl Acetate in a Double Jacket Reactor and Simultaneous Rearrangement/Dehydration Using CrO₃—PVPy

### Examples 5 and 6

### Apparatus:

Double jacket vessel, cylindrical, with tempered outer jacket, inner diameter 45 mm, total volume 150 mL (reaction volume approx. 24 mL, corresponding to approx. 18 mm filling height), illuminated from below by 24 LEDs with a wavelength of 405 nm, total radiometric power 27 W, impeller stirrer.

### Experiment:

26.3 g (205.27 mmol, 1 eq) of isoprenyl acetate (IPA) were placed in a glass bottle, 1.22 g (6.28 mmol, 0.031 eq) of dimethyl phthalate were added as an internal NMR standard, and 9.8 mg (15.9 μmol, 0.000078 eq) of the photosensitizer tetraphenylporphyrin (TPP) were dissolved in the mixture as a photosensitizer. The reaction mixture was poured into the temperature-controlled double-jacketed vessel together with 5 g of CrO₃—PVPy (loading about 12.5% by weight of CrO₃, relative to the total weight of the supported catalyst) and stirred at 800 rpm. At 30 °C, the solution was irradiated from below for a period of 4 hours while 2 L/h (example 7) or 1.5 L/h (example 8) of oxygen were introduced into the solution. At the end of the experiment, the reaction mixture was analysed without further workup. The results are summarized in Table 3.

TABLE 3

| | | Conversion | Yield I.a^{#} | | Yield I.b^{##} | |
|---|---|---|---|---|---|---|
| No. | O₂ [L/h] | IPA [%] | [g] | [%]¹ | [g] | [%]¹ |
| 5 | 1.5 | 16.5 | 2.6 | 8.9 | 0.9 | 3.0 |
| 6 | 2.0 | 20.6 | 3.0 | 10.1 | 1.1 | 3.8 |

¹Yield relative to the amount of IPA as used in the reaction (here 205.27 mmol)

### Example 7

### Apparatus:

Double jacketed vessel, cylindrical, with tempered outer jacket, inner diameter 45 mm, total volume 150 mL (reaction volume approx. 24 mL, corresponding to approx. 18 mm filling height), illuminated from below by 24 LEDs with a wavelength of 405 nm, total radiometric power 27 W, impeller stirrer.

### Experiment:

23.7 g (184.8 mmol, 1 eq) of isoprenyl acetate (IPA) were placed in a glass bottle, 1.22 g (6.28 mmol, 0.034 eq) of dimethyl phthalate was added as an internal NMR standard, and 10.0 mg (16.3 μmol, 0.000088 eq) of the photosensitizer tetraphenylporphyrin (TPP) were dissolved in the mixture. In addition, 2.62 g (17.0 mmol, 0.09 eq) of carbon tetrachloride were added. The reaction mixture was poured into the temperature-controlled double-jacketed vessel together with 3.7 g of CrO₃—PVPy (loading about 12.5% by weight of CrO₃, relative to the total weight of the supported catalyst) and stirred at 800 rpm. At 30 °C, the solution was irradiated from below for a period of 4 hours while 1.5 L/h of oxygen were introduced into the solution. At the end of the experiment, the reaction mixture was analysed without further workup. The results are summarized in Table 4.

TABLE 4

| | | |
|---|---|---|
| Conversion | Yield I.a^{#} | Yield I.b^{##} |

| No. | IPA [%] | [g] | [%]¹ | [g] | [%]¹ |
|---|---|---|---|---|---|
| 7 | 19.1 | 2.5 | 9.7 | 0.9 | 3.3 |

¹Yield relative to the amount of IPA as used in the reaction (here 184.8 mmol)

The yields given above are relative to the amount of the starting compound (IPA) as used in the reaction. Since the reactions are carried out so that only a rather small portion of the starting compound is reacted (and the remainder can further serve as dispersing medium), only yields based on the amount of the reacted starting material reflect the effectiveness of the reaction. These can be calculated by relating the yields of I.a and I.b to the converted amount of IPA (not shown in the above tables).

### 4. Photooxidation of Isoprenyl Acetate to Isoprenyl Acetate Hydroperoxide

### Example 8

Apparatus: Corning® G1 photoreactor (5 tempered G1 plates, layer thickness approx. 1 mm, each irradiated on both sides by LEDs, a total of 200 LEDs with a wavelength of 405 nm, total radiometric power 195 W), 100 mL miniplant reactor, impeller stirrer, gear pump.

### Experiment:

In the 100 mL miniplant reactor, a mixture of 135.0 g (1053.3 mmol, 1 eq) of isoprenyl acetate (IPA) and 6.22 g (32.0 mmol, 0.03 eq) of dimethyl phthalate as internal NMR standard was prepared and 15 mg (24.7 μmol, 0.000023 eq) of tetraphenylporphyrin as photosensitizer was dissolved in said mixture. The reaction solution was stirred at 100 rpm and pumped over the Corning® reactor in a circuit. At the temperature indicated in Table 5, the solution was irradiated for a period of 6 hours while 3 L/h of oxygen were introduced into the Corning® reactor at 1.7-2.4 bar. The reaction was terminated distinctly before complete conversion of isoprenyl acetate (the conversion rate for the experiment is listed in Table 5). At the end of the experiment, the reaction mixture was analysed without further workup. The results are summarized in Table 5.

TABLE 5

| | | | Yield¹ | |
|---|---|---|---|---|
| | | Conversion | hydroperoxide⁺ [%] | |
| No. | T [°C] | IPA [%] | [g] | [%]¹ |
| 14 | 30 | 29.7 | 24.6 | 14.6 |

¹Yield relative to the amount of IPA as used in the reaction (here 1053.3 mmol)
⁺compound III.a wherein R¹ = —C(O)CH₃

III.a⁺: ¹H-NMR (500 MHz, CDCl₃): δ=5.19 (1H), 5.10 (1H), 4.47 (2H), 4.26 (2H), 2.47 (2H), 2.06 (3H)

¹³C-NMR (125 MHz, CDCl₃): δ=171.19 (s), 140.75 (s), 116.58 (t), 79.82 (t), 62.69 (t), 32.36 (t), 22.34 (q)

Moreover, compound III.c wherein R¹=—C(O)CH₃ was identified, which is presumed to be the result of the further photooxidation of compound III.b taking place in competition with the conversion of III.b to I.b, or directly of (double) photooxidation of II.a:

III.c⁺: ¹H-NMR (700 MHz, CDCl₃): δ=5.45 (1H), 5.41 (1H), 4.71 (1H), 4.55 (2H), 4.39 (2H), 2.09 (3H)

¹³C-NMR (175 MHz, CDCl₃): δ=170.6 (s), 139.8 (s), 120.2 (t), 83.3 (d), 77.8 (t), 62.7 (t), 21.1 (q)

### 5. Photooxidation of Isoprenol to Isoprenol Hydroperoxide

### Example 9

Apparatus: Corning® G1 photoreactor (5 tempered G1 plates, layer thickness approx. 1 mm, each irradiated on both sides by LEDs, a total of 200 LEDs with a wavelength of 610 nm, total radiometric power 83 W), 100 mL miniplant reactor, impeller stirrer, gear pump.

### Experiment:

In the 100 mL miniplant reactor, a mixture of 135.0 g (1567.4 mmol, 1 eq) of isoprenol (IP; compound II.a wherein R¹ is H) and 9.13 g (47.0 mmol, 0.03 eq) of dimethyl phthalate as the internal NMR standard was prepared, and 28.3 mg (84 μmol, 0.000054 eq) of methylene blue monohydrate as a photosensitizer was dissolved in said mixture. The reaction solution was stirred at 300 rpm and pumped over the Corning® reactor at 100 mL/min in a circuit. At 30 °C, the solution was irradiated for a period of 4 hours while 3 L/h of oxygen were introduced into the Corning® reactor at 5 bar. During the reaction, a further 63.5 mg (188 μmol) of photosensitizer were added in portions The reaction was terminated distinctly before complete conversion of isoprenol (the conversion rate for the experiment is listed in Table 6). At the end of the experiment, the reaction mixture was analysed without further workup. The results are summarized in Table 6.

TABLE 6

| | | | Yield¹ hydro- | | | |
|---|---|---|---|---|---|---|
| | | Conversion IP | peroxide⁺⁺ [%] | | Yield¹ I.a^{###} [%] | |
| No. | T [°C] | [%] | [g] | [%]¹ | [g] | [%]¹ |
| 15 | 30 | 11.7 | 7.3 | 3.9 | 0.1 | 0.1 |

¹Yield relative to the amount of IP as used in the reaction (here 1567.4 mmol)
⁺⁺compound III.a wherein R¹ = H
^{###}Compound I.a wherein R¹ = H
III.a⁺⁺: ¹H-NMR (400 MHz, CDCl₃): δ = 5.24 (1H), 5.16 (1H), 4.47 (2H), 3.86 (2H), 2.41 (2H)

### Example 10

Apparatus: Double jacket vessel, cylindrical, with tempered outer jacket, inner diameter 45 mm, total volume 150 mL (reaction volume approx. 38 mL, corresponds to approx. 24 mm filling height), illuminated from below by 24 LEDs with a wavelength of 405 nm, total radiometric power 27 W, impeller stirrer.

### Experiment:

In a 50 mL glass vial, a mixture of 30.0 g (348.3 mmol, 1 eq) of isoprenol (IP), 1.39 g (7.2 mmol, 0.021 eq) of dimethyl phthalate as internal NMR standard and 1.5 g (12.5 mmol, 0.036 eq) of chloroform was prepared, and 4.6 mg (7.5 μmol, 0.000021 eq) of tetraphenylporphyrin as photosensitizer was dissolved in said mixture. The reaction solution was poured into the temperature controlled double jacket vessel and stirred at 800 rpm. At 10 °C, the solution was irradiated for a period of 5 hours while 2 L/h of oxygen were introduced into the solution. During the reaction, a further 8.2 mg (13.3 μmol) of photosensitizer were added in portions. The reaction was terminated distinctly before complete conversion of isoprenol (the conversion rate for the experiment is listed in Table 7). At the end of the experiment, the reaction mixture was analysed without further workup. The results are summarized in Table 7.

TABLE 7

| | | | Yield¹ hydro- | | | |
|---|---|---|---|---|---|---|
| | | Conversion IP | peroxide⁺⁺ [%] | | Yield¹ I.a^{#} [%] | |
| No. | T [°C] | [%] | [g] | [%]¹ | [g] | [%]¹ |
| 16 | 30 | 23.0 | 3.6 | 8.6 | 0.1 | 0.3 |

¹Yield relative to the amount of IP as used in the reaction (here 348.3 mmol)

1.-18. (canceled)

19. A method for preparing a compound of the formula I.a or of the formula I.b or a mixture thereof or a stereoisomer of the compound I.a or I.b or a mixture of different stereoisomers of the compound I.a and/or I.b or a mixture of different compounds I.a and/or I.b 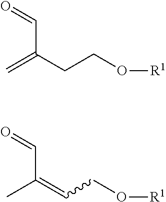
wherein
R¹ is hydrogen or —C(═O)R²; and
R² is C₁-C₂₀-alkyl,
the method comprising:
(i) providing a reaction mixture comprising a compound of the formula II.a 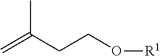
wherein R¹ is as defined above;
a photosensitizer and optionally a transition metal catalyst;
(ii) passing an oxygen-containing gas through the reaction mixture provided in step (i) and simultaneously irradiating the reaction mixture with light;
(iii) if in step (i) no transition metal catalyst has been provided, bringing the reaction mixture of or obtained in step (ii) into contact with a transition metal catalyst;
(iv.1) optionally, after completion of the reaction, isolating the one or more compounds (I.a) or (I.b) obtained in step (ii) or (iii); and
(v.1) optionally hydrolysing the one or more compounds (I.a) or (I.b) isolated in step (iv.1) to compounds (I.a) or (I.b) wherein R¹ is hydrogen;
or
(iv.2) optionally, hydrolysing the reaction mixture obtained in step (ii) or (iii); and
(v.2) optionally, isolating the one or more compounds (I.a) or (I.b) obtained in step (iv.2).

20. The method according to claim 19, wherein R² is C₁-C₄-alkyl or n-C₁₅-alkyl.

21. The method according to claim 20, wherein R² is C₁-C₄-alkyl.

22. The method according to claim 19, wherein the transition metal catalyst used in step (i) or (iii) is a heterogeneous catalyst, wherein the support material is selected from the group consisting of carbon, alumina, silica, zeolite and organic polymers.

23. The method according to claim 22, wherein the heterogeneous transition metal catalyst comprises at least one salt or oxide of a transition metal of group 4 to 12 of the Periodic Table of Elements.

24. The method according to claim 23, wherein the salt or oxide of the transition metal is a C₂-C₈-carboxylate, halide, nitrate, phosphate, sulfate, chlorate, perchlorate or oxide of a transition metal of the 4^{th} period of group 4 to 12 of the Periodic Table of Elements, or is a chromate, chlorochromate, dichromate or permanganate.

25. The method according to claim 24, wherein the salt or oxide of the transition metal is Cr(VI) oxide (CrO₃).

26. The method according to claim 19, wherein the transition metal catalyst used in step (i) or (iii) is a supported heterogeneous catalyst, wherein the support material is an organic polymer, wherein the organic polymer is selected from the group consisting of vinylpyridine homo- and copolymers, N-vinylpyrrolidine homo- and copolymers, styrene homo- and copolymers, polyurethanes, acrylate homo- and copolymers and methacrylate homo- and copolymers.

27. The method according to claim 26, wherein organic polymer is selected from the group consisting of 2-vinylpyridine homo- and copolymers, 4-vinylpyridine homo- and copolymers and N-vinylpyrrolidine homo- and copolymers.

28. The method according to claim 19, wherein the transition metal is Cr(VI) oxide (CrO₃) on polyvinylpyridines.

29. The method according to claim 19, wherein the photosensitizer is selected from the group consisting of fluorescein, eosin, rose bengal, erythrosine, tetraphenylporphyrin, cobalt-tetraphenylporphyrin, zinc-tetraphenylporphyrin, hematoporphyrin, rhodamine B, basacryl brilliant red, methyl violet, methylene blue, fullerene C₆₀, fullerene C₇₀, graphene, carbon nanotubes, Ru(bpy)₃²⁺ salts, Ru(phen)₃²⁺ salts, cercosporin, hypocrellin-A and mixtures thereof.

30. The method according to claim 29, wherein the photosensitizer is selected from the group consisting of tetraphenylporphyrin, zinc-tetraphenylporphyrin and Ru(bpy)₃²⁺ salts.

31. The method according to claim 19, wherein in step (ii) the reaction mixture is irradiated with light in the wavelength range of from 350 to 800 nm.

32. The method according to claim 29, wherein the photosensitizer is tetraphenylporphyrin, cobalt-tetraphenylporphyrin or zinc-tetraphenylporphyrin and in step (ii) the reaction mixture is irradiated with light in the wavelength range of from 400 to 430 nm; or the photosensitizer is methylene blue and in step (ii) the reaction mixture is irradiated with light in the wavelength range of from 600 to 620 nm, or the photosensitizer is a Ru(bpy)₃²⁺ salt or a Ru(phen)₃²⁺ salt and in step (ii) the reaction mixture is irradiated with light in the wavelength range of from 450 to 480 nm.

33. The method according to claim 19, wherein the oxygen-containing gas used in step (ii) is selected from the group consisting of oxygen, air and mixtures of oxygen and nitrogen containing oxygen in a range of from 1 to 99% by weight, relative to the total weight of the mixture.

34. The method according to claim 19, wherein steps (ii) and (iii) are carried out neat; or
steps (ii) and (iii) are carried out in the presence of a chlorinated C₁-C₂-alkane, wherein the molar ratio of the compound (II.a) provided in step (i) to the chlorinated C₁-C₂-alkane is of from 50:1 to 1:1.5.

35. The method according to claim 27, wherein the organic polymer is selected from the group consisting of poly(2-vinylpyridine), poly(4-vinylpyridine), copolymers of 2-vinylpyridine and methyl methacrylate, and poly(N-vinylpyrrolidine).

36. A hydroperoxide compound of the formula III.a, III.b or III.c or a stereoisomer of the compound of the formula III.a, III.b or III.c or a mixture of different stereoisomers of the compound III.a, III.b and/or III.c or a mixture of different compounds III.a, III.b and/or III.c 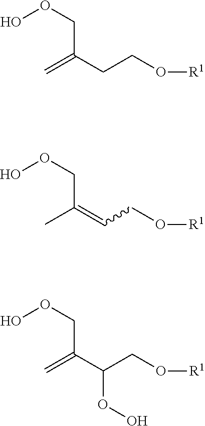
wherein
in compounds III.a R¹ is hydrogen or —C(═O)R²; and R² is C₁-C₂₀-alkyl;
in compounds III.b R¹ is —C(═O)R²; and R² is C₁-C₂₀-alkyl; and
in compounds III.c R¹ is hydrogen or —C(═O)R²; and R² is C₁-C₂₀-alkyl;
a hydroperoxide compound of the formula III.a or III.b or a stereoisomer of the compound of the formula III.a or III.b or a mixture of different stereoisomers of the compound III.a and/or III.b or a mixture of different compounds III.a and/or III.b.
